(19) United States
(12) Patent Application Publication
(10) Pub. No.: US 2016/0030404 A1
(43) Pub. Date: Feb. 4, 2016

(54) Therapeutic Agents and Methods for the Treatment of DNA Repair Deficiency Disorders

(71) Applicant: THE REGENTS OF THE UNIVERSITY OF CALIFORNIA, Oakland, CA (US)

(72) Inventors: Robert H. Schiestl, Encino, CA (US); Yelena O. Rivina, Sherman Oaks, CA (US); Michael Davoren, Los Angeles, CA (US)

(21) Appl. No.: 14/776,914

(22) PCT Filed: Mar. 13, 2014
(86) PCT No.: PCT/US2014/025929

§ 371 Date: Sep. 15, 2015

Related U.S. Application Data
(60) Provisional application No. 61/801,169, filed on Mar. 15, 2013.

(51) Int. Cl. A61K 31/437; A61K 31/381

(52) U.S. Cl. CPC A61K 31/437; A61K 31/381

(57) ABSTRACT

The present invention provides compounds, compositions, its, and methods which are effective for mitigating, treating, or ameliorating a DNA repair-deficiency disorder or a symptom of a DNA repair-deficiency disorder. The compounds, compositions, kits, and methods are also effective for modulating a level or activity of a DNA repair enzyme or a level or activity of gene encoding a DNA repair enzyme.

### CROSS-REFERENCE TO RELATED APPLICATIONS

This application claims the benefit of U.S. Provisional Application No. 61/801,169, filed on Mar. 15, 2013; the entire content of this application is incorporated herein in its entirety by this reference.

### STATEMENT REGARDING FEDERALLY SPONSORED RESEARCH

This invention was made with Government support under Grant No. AI067769, awarded by the National Institutes of Health. The U.S. Government has certain rights in the invention. The statement is included solely to comply with 37 C.F.R. §401.14(a)(f)(4) and should not be taken as an assertion or admission that the application discloses and/or claims only one invention.

### FIELD OF THE INVENTION

The present invention generally relates to compounds and methods for modulating the level or activity of a DNA repair enzyme or a level or activity of gene encoding a DNA repair enzyme, compositions comprising these compounds and methods of making and using the same. Agents described herein are useful for mitigating, ameliorating, or treating a DNA repair deficiency disorder or a symptom of DNA repair deficiency disorder.

### BACKGROUND OF THE INVENTION

The human genome is continuously exposed to potentially deleterious genotoxic events from both endogenous sources, resulting from cellular metabolism or routine errors in DNA replication and recombination and reactive oxygen species, and exogenous sources such as ionizing radiation, sunlight, ultraviolet light (UV), and chemical mutagens. (Mirzayans et al. The Open Cancer Journal (2008) 2:42-52). Genome integrity and cellular homeostasis are maintained through elegant DNA surveillance networks that serve to recognize the DNA damage and facilitate DNA repair. These networks are complex signal transduction pathways that coordinate cell-cycle checkpoints and DNA repair processes to eliminate DNA damage, as well as invoking pathways such as sustained growth arrest, accelerated senescence and apoptotic cell death to eliminated injured cells from the proliferating population. Failure of cells to properly activate these pathways following endogenous and exogenous genotoxic stress may lead to the development of genomic instability, DNA repair deficiency, and the emergence of malignant cells. It is therefore not surprising that defects in key components in the DNA surveillance networks are the underlying cause of numerous debilitating human genetic disorders that are characterized by genomic instability, DNA repair deficiency, premature aging, cancer susceptibility, and anemia.

Thus, there is a need to identify drugs that can upregulate DNA surveillance networks to be used in the prophylaxis, mitigation, amelioration, and treatment of DNA repair deficiency disorder or a symptom associated with a DNA repair deficiency disorder.

### SUMMARY OF THE INVENTION

One aspect of the present invention provides a compound effective for modulating a level or activity of a DNA repair enzyme or a level or activity of a gene encoding a DNA repair enzyme.

In one aspect, agents described herein are useful for mitigating, treating, or ameliorating a DNA repair deficiency disorder or a symptom associated with a DNA repair deficiency disorder.

In some embodiments, the DNA repair deficiency disorder is selected from Ataxia Telangiectasia (A-T), Xeroderma Pigmentosum (XP), Fanconi's Anemia (FA), Li Fraumeni, Nijmegen breakage syndrome (NBS), A-T-like disorder (ATLD), Werner's syndrome, Bloom's syndrome, Rothmund-Thompson syndrome, Cockayne's syndrome (CS), or Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, Ataxia with oculomotor apraxia 2, Diamond-Blackfan anemia, Rapadilino syndrome, Turcot Syndrome, Seckle Syndrome, Lynch syndrome, NBS-like syndrome, and RIDDLE Syndrome.

Another aspect of the present invention provides a method for modulating a level or activity of a DNA repair enzyme or a level or activity of gene encoding a DNA repair enzyme. The method comprises: (a) administering to a subject in need of modulating a level or activity of a DNA repair enzyme or a level or activity of gene encoding a DNA repair enzyme a pharmaceutically effective amount of a composition comprising: (i) a compound having a structure of Formula 1; or (ii) a compound having a structure of Formula 2; or (iii) single stereoisomer, mixtures of stereoisomers, pharmaceutically acceptable salts or prodrugs of (i) and/or (ii).

In some embodiments, the DNA repair enzyme is selected from ATM, MRE11, NBN, RAD50, APEX1 (APEX nuclease 1), DDB1 (Damage-specific DNA binding protein 1), XRCC4, SMC3, SMC2, SMC4, or condensin.

Useful compounds for modulating level or activity of a DNA repair enzyme or a level or activity of a gene encoding a DNA repair enzyme are described in PCT/US2011/046451 and U.S. publication no. 2013/0231518, which is incorporated herewith by reference in its entirety for all purposes, and particularly for the compounds it discloses.

Another aspect of the present invention provides a compound effective for mitigating, treating or ameliorating a DNA repair deficiency disorder or a symptom associated with a DNA repair deficiency disorder. The compound can be a synthetic compound or a natural product in a substantially purified form. The compound also includes a pharmaceutically acceptable salt thereof, a prodrug thereof, a hydrate thereof, a solvate thereof, or a polymorphic crystal thereof.

In some embodiments, the compound comprises a structure of Formula I or Formula II:

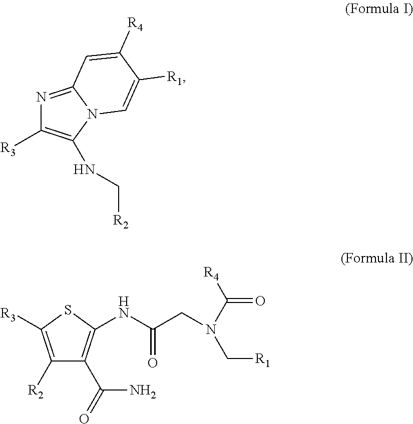

where:

in Formula I, each R₁, R₂ and R₃ are independently hydrogen, straight chain or branched C1-C20 alkyl, alkenyl, or alkynyl, which is substituted or unsubstituted, cyclo alkyl, cyclo alkenyl, heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, —BH₂, alkoxy, or acyl groups along with a list of the following exemplary substitutions:

Each R₁ independently=one or more of the following NH₂, OH, OMe, Me, H, CH₂OH, BH₂, SMe,

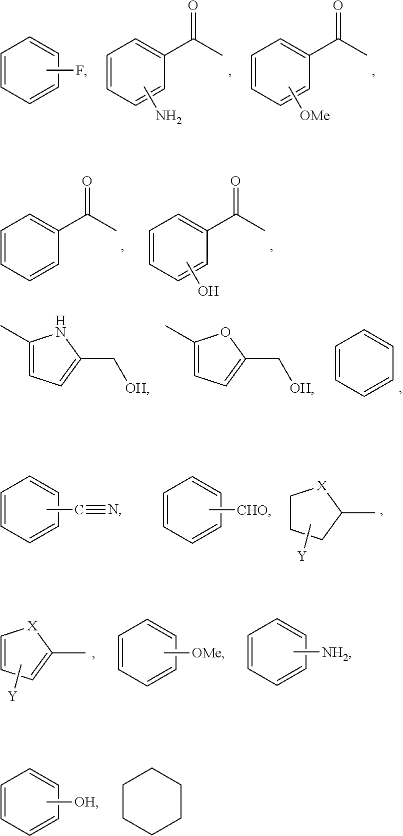

X=S, HN, O, BH, CH₂:

Y=NH₂, OH, OMe, Me, H, CH₂OH, BH₂, SeMe, SMe

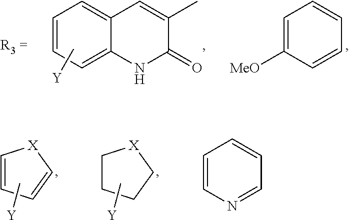

X=S, NH, O, BH, CH₂

Y=NH₂, OH, OMe, Me, H, CH₂OH, BH₂, SeMe, SMe

and in Formula II, R₁, R₂, R₃, and R₄ are independently hydrogen, straight chain or branched C1-C20 alkyl, alkenyl, or alkynyl, which is substituted or unsubstituted, cyclo alkyl, cyclo alkenyl, heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, alkoxy, or acyl groups along with a list of the following exemplary substitutions:

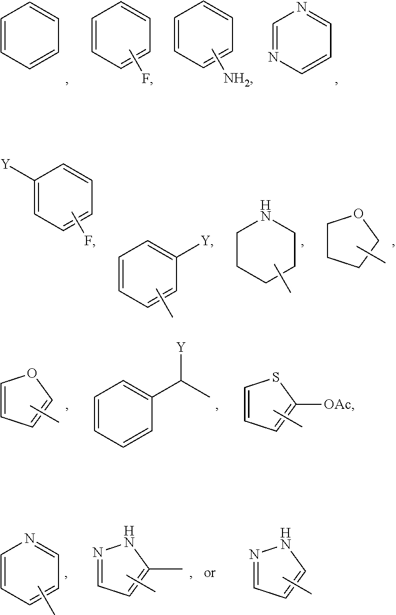

### Y=CH₃, OH; and R₂=R₃=and are Ch₃, OH, O, SH, H, NH₂, or

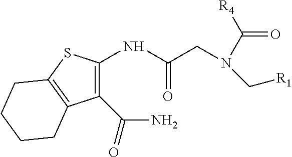

In some embodiments, the compound has a Tanimoto coefficient at least 0.7 or higher based on a compound of Formula IA, Formula IIA or Formula IIB:

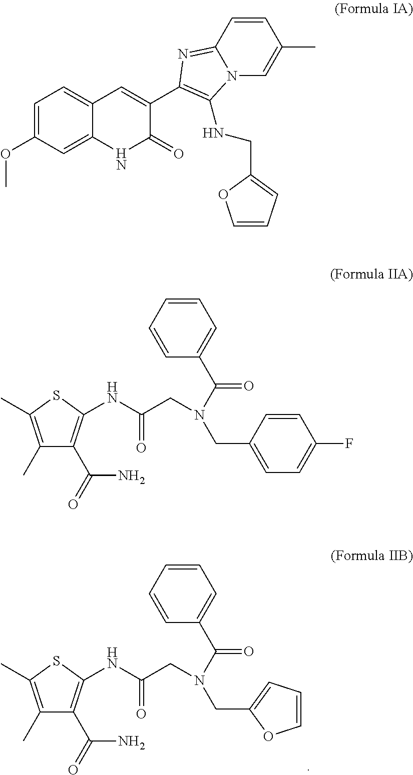

In some embodiments, the compound is selected from

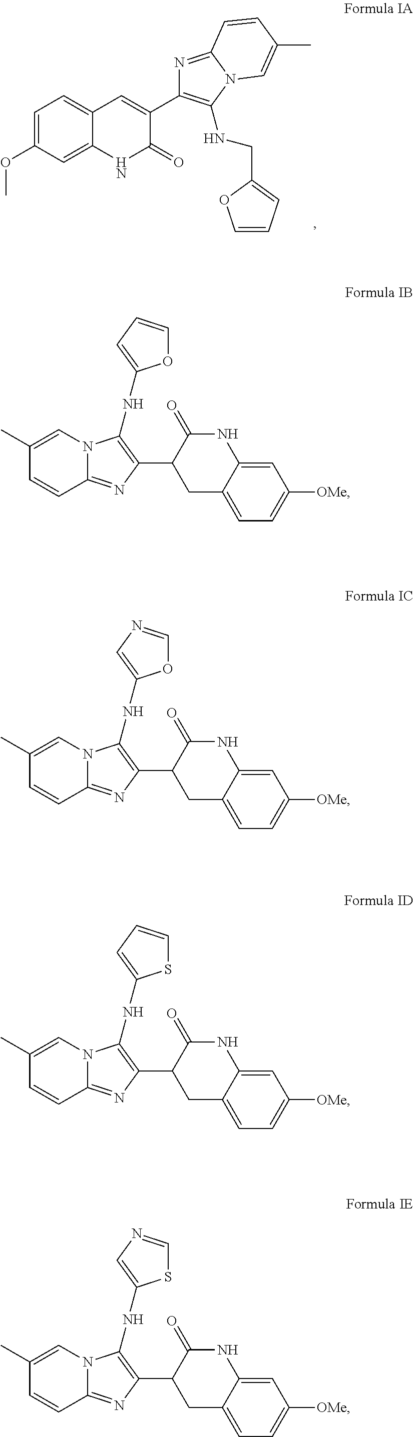

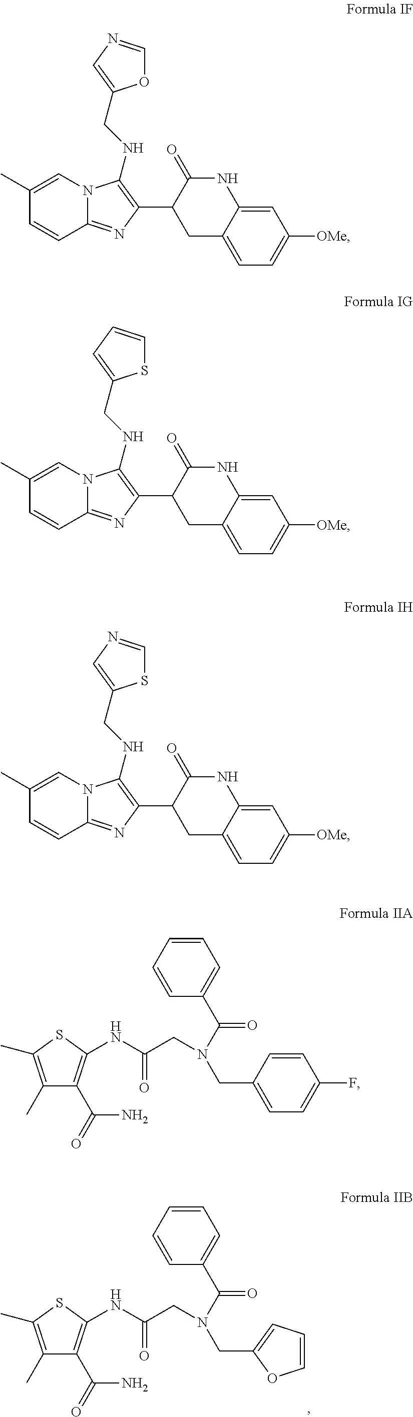 [SMILES: COc1ccc2c(c1)NC(=O)C(c1nc3ccc(C)cn3c1NCc1cnco1)C2 ; COc1ccc2c(c1)NC(=O)C(c1nc3ccc(C)cn3c1NCc1cccs1)C2 ; COc1ccc2c(c1)NC(=O)C(c1nc3ccc(C)cn3c1NCc1cncs1)C2 ; Cc1sc(NC(=O)CN(Cc2ccc(F)cc2)C(=O)c2ccccc2)c(C(N)=O)c1C ; Cc1sc(NC(=O)CN(Cc2ccco2)C(=O)c2ccccc2)c(C(N)=O)c1C]

or a combination of the compounds of Formula IA-IH, Formula IIA, or Formula IIB.

In some embodiments, the compound of the various embodiments herein may be a pharmaceutically acceptable salt thereof, or a prodrug thereof.

Another aspect of the present invention provides a composition which comprises a compound of the various embodiments disclosed herein. The composition comprises the compound in an amount effective for mitigating, treating, or ameliorating a DNA repair deficiency disorder or a symptom associated with a DNA repair deficiency disorder.

In some embodiments, the composition can further optionally include at least one other therapeutic agent.

In some embodiments, the composition further comprises an excipient.

In some embodiments, the composition of various embodiments disclosed herein further comprises a pharmaceutically acceptable carrier.

The compounds and compositions disclosed herein can be formulated into a formulation for local delivery or systemic delivery. In some embodiments, the composition is formulated into a formulation for oral administration, parenteral administration (e.g., intravenous), injection, topical administration, implant, or pulmonary administration.

A further aspect of the present invention provides a method of screening for a compound effective as a mitigating agent. The method comprises:

generating a screening system capable of screening a compound against a DNA repair deficiency disorder;

subjecting a compound to the screening, and

identifying a candidate compound as effective if the compound significantly reduces genetic instability, induces DNA repair, restores proliferative regulation in stem cells, restores cell viability in bone marrow, increases the number of red blood cell progenitors, or increase in the production of erythrocytes as compared to a control.

In some embodiments of the method, the compound has a structure of Formula I or Formula II.

The method comprises providing a compound which is effective for mitigating, treating, or ameliorating a DNA repair deficiency disorder or a symptom associated with a DNA repair deficiency disorder, and forming the composition of the various embodiments disclosed herein. The compound is as in the various embodiments disclosed herein.

A further aspect of the present invention provides a method of treating, preventing, or ameliorating a condition. The method comprises administering to a subject a compound or a composition according to the various embodiments of disclosed herein.

In some embodiments, the compound is included in a composition.

In some embodiments, the composition further includes an optional second agent.

It is contemplated that all embodiments described herein, including those described under different aspects of the invention, can be combined with one another where not specifically prohibited.

### BRIEF DESCRIPTION OF THE DRAWINGS

FIG. 1 schematically depicts the benefit of administration of Yel002. ATM −/− mice receiving weekly injections of Yel002 have much smaller observed rates of lymphoma and AT associated deaths. Mice treated with weekly injection of 75 mg/kg Yel002 had a medium increased survival of 16 weeks compared to the untreated mice which is highly significant. This translates into about 12 years of human life, which would be a tremendous improvement in the life expectancy of AT patients. Details are described in Example 1.

FIG. 2 shows Diamond Blackfan Anemia zebrafish embryos. Wild-type embryo (top slide). Diamond Blackfan Anemia embryos shows lack of erythrocytes (left and right middle slides). Diamond Blackfan Anemia embryos treated with Yel002 show reappearance of erythrocytes (left and right bottom slides).

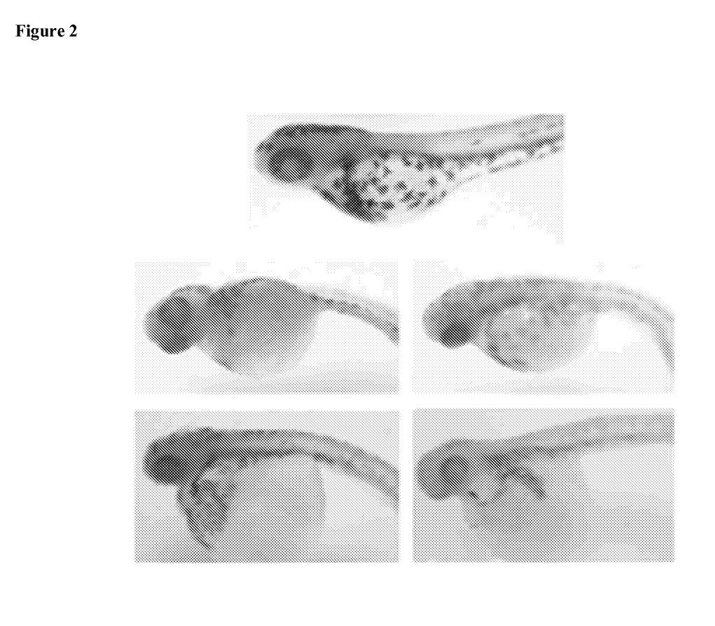

### DETAILED DESCRIPTION OF THE INVENTION

### I. Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. The term “consisting essentially of means that the composition, method or structure may include additional ingredients, steps and/or parts, but only if the additional ingredients, steps and/or parts do not materially alter the basic and novel characteristics of the claimed composition, method or structure. No language in the specification should be construed as indicating any non-claimed element essential to the practice of the invention.

The terms “a” and “an” and “the” and similar referents used in the context of describing the invention (especially in the context of the following claims) are to be construed to cover both the singular and the plural, unless otherwise indicated herein or clearly contradicted by context.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximate”) one particular value, and/or to “about” (or “approximate”) another particular value. When such a range is expressed, another embodiment includes from the one particular value and/or to the other particular value. Similarly, when values are expressed as approximations, by use of the antecedent “about” or “approximate” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that is “less than or equal to the value” or “greater than or equal to the value” possible ranges between these values are also disclosed, as appropriately understood by the skilled artisan. For example, if the value “10” is disclosed the “less than or equal to 10” as well as “greater than or equal to 10” is also disclosed.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e. g. “such as”) provided herein is intended merely to better illuminate the invention and does not pose a limitation on the scope of the invention otherwise claimed.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to cat-boxy orientation.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety. Illustrations are for the purpose of describing a preferred embodiment of the invention and are not intended to limit the invention thereto.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, the term “about” refers to a range of values of plus or minus 10% of a specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, administration “in combination” refers to both simultaneous and sequential administration of two or more compositions. Concurrent or combined administration, as used herein, means that two or more compositions are administered to a subject either (a) simultaneously, or (b) at different times during the course of a common treatment schedule. In the latter case, the two or more compositions are administered sufficiently close in time to achieve the intended effect.

The terms “agent” and “compound” are used interchangeably herein.

As used herein, the term “biologically active” when referring to an agent is art recognized and refers to a form of an agent that allows for it, or a portion of the amount of agent administered, to be absorbed by, incorporated to, or otherwise physiologically available to a subject or patient to whom it is administered.

As used herein, “biological sample” means a sample of biological tissue or fluid that contains nucleic acids or polypeptides. Such samples are typically from humans, but include tissues isolated from non-human primates, or rodents, e.g., mice, and rats. Biological samples may also include sections of tissues such as biopsy and autopsy samples, frozen sections taken for histological purposes, cerebral spinal fluid, blood, plasma, serum, sputum, stool, tears, mucus, hair, skin, etc. Biological samples also include explants and primary and/or transformed cell cultures derived from patient tissues. A “biological sample” also refers to a cell or population of cells or a quantity of tissue or fluid from an animal. Most often, the biological sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of blood, serum, saliva, cerebral spinal fluid or urine, that can be used to measure expression level of a polynucleotide or polypeptide. Numerous types of biological samples can be used in the present invention, including, but not limited to, a tissue biopsy or a blood sample. As used herein, a “tissue biopsy” refers to an amount of tissue, such as a lung tissue, removed from an animal, preferably a human, for diagnostic analysis. “Tissue biopsy” can refer to any type of biopsy, such as needle biopsy, fine needle biopsy, surgical biopsy, etc.

As used herein, the term “decreased expression” refers to the level of a gene expression product that is lower and/or the activity of the gene expression product is lowered. Preferably, the decrease is at least 20%, more preferably, the decrease is at least 30%, at least 40%, at least 50%, at least 60%, at least 70%, at least 80%, or at least 90% and most preferably, the decrease is at least 100%, relative to a control.

Synonyms of the term “determining” are contemplated within the scope of the present invention and include, but are not limited to, detecting, measuring, assaying, or testing for the presence, absence, amount or concentration of a molecule, a label, or a small molecule of the invention and the like. The term refers to both qualitative and quantitative determinations.

As used herein, “determining the effect” or “determining the functional effect” means assaying for an agent that increases or decreases a parameter that is indirectly or directly under the influence of the agent, e.g., functional, enzymatic, physical and chemical effects. Such effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of a gene, such as a gene encoding a DNA repair enzyme, measuring binding activity, measuring cellular proliferation, measuring apoptosis, measuring subcellular localization of a polypeptide, such as a DNA repair enzyme, or the like. Determination of the functional effect of an agent on a disease, disorder, or other pathology can also be performed using assays known to those of skill in the art such as in vitro assays, e.g., cellular proliferation; growth factor or serum dependence; mRNA and protein expression in cells, and other characteristics of cells. The effects can be evaluated by many means known to those skilled in the art, e.g., microscopy for quantitative or qualitative measures of alterations in morphological features, measurement of changes in RNA or protein levels, measurement of RNA stability, identification of downstream or reporter gene expression (CAT, luciferase, B-gal, GFP and the like), e.g., via chemiluminescence, fluorescence, colorimetric reactions, antibody binding, inducible markers, ligand binding assays, apoptosis assays, measuring the production of acetyl-CoA and AMP, and the like.

As used herein, “disorder”, “disease” or “pathological condition” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information. A preferred “disorder”, “disease” or “pathological condition” amenable to prevention and/or treatment using compositions and methods described herein is a DNA repair deficiency disorder.

As used herein, the term “DNA repair deficiency disorder” refers to a disorder in a subject in which one or more components of the DNA repair pathway(s) is underexpressed, mutated, or less functional than the same component in a wild-type organism. A DNA repair deficiency disorder may refer to a subject in which at least a cell has a mutation. Examples of DNA repair deficiency disorders include, but are not limited to, Ataxia Telangiectasia (A-T), Xeroderma Pigmentosum (XP), Fanconi's Anemia (FA), Li Fraumeni syndrome, Nijmegen breakage syndrome (NBS), A-T-like disorder (ATLD), Werner's syndrome, Bloom's syndrome, Rothmund-Thompson syndrome, Cockayne's syndrome (CS), Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, Ataxia with oculomotor apraxia 2, Diamond Blackfan anemia, Rapadilino syndrome, Turcot Syndrome, Seckle Syndrome, Lynch syndrome, NBS-like syndrome, and RIDDLE Syndrome.

As used herein, the term “DNA repair enzyme” refers to a polypeptide involved in repairing a mutation within a nucleic acid. A DNA repair enzyme can be from a prokaryote or a, eukaryote. A preferred DNA repair enzyme is from a eukaryote. Even more preferred is a mammalian DNA repair enzyme. Most preferred is a human DNA repair enzyme. DNA repair enzymes are known in the art.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting or transit that can be associated with the administration of the pharmaceutical composition. With respect to “effective amount”, “effective dose”, “sufficient amount”, “amount effective to”, “therapeutically effective amount” of a probiotic microorganism, the dosing range varies with the pharmaceutical composition used, the route of administration and the potency of the particular pharmaceutical composition.

“Functional effects” include in vitro, in vivo, and ex vivo activities.

The terms “individual,” “subject,” “host,” and “patient,” used interchangeably herein, refer to a mammal, including, but not limited to, murines, simians, felines, canines, equines, bovines, mammalian farm animals, mammalian sport animals, and mammalian pets and humans. Preferred is a human.

As used herein, “in vitro” means outside the body of the organism from which a cell or cells is obtained or from which a cell line is isolated.

As used herein, “in vivo” means within the body of the organism from which a cell or cells is obtained or from which a cell line is isolated.

As used herein, “level of an mRNA” in a biological sample refers to the amount of mRNA transcribed from a gene that is present in a cell or a biological sample. The mRNA generally encodes a functional protein, although mutations may be present that alter or eliminate the function of the encoded protein. A “level of mRNA” need not be quantified, but can simply be detected, e.g., a subjective, visual detection by a human, with or without comparison to a level from a control sample or a level expected of a control sample. A preferred mRNA is a mRNA transcribed from a gene encoding a DNA repair enzyme.

As used herein, “level of a polypeptide” in a biological sample refers to the amount of polypeptide translated from an mRNA that is present in a cell or biological sample. The polypeptide may or may not have protein activity. A “level of a polypeptide” need not be quantified, but can simply be detected, e.g., a subjective, visual detection by a human, with or without comparison to a level from a control sample or a level expected of a control sample. A preferred polypeptide is a DNA repair enzyme.

As used herein, “mammal” or “mammalian” means or relates to the class mammalia including the orders carnivore (e.g., dogs and cats), rodentia (e.g., mice, guinea pigs, and rats), and primates (e.g., humans, chimpanzees, and monkeys.

As used herein, the terms “modulate,” “modulation,” “modulating” and grammatical equivalents thereof are art-recognized and refer to up-regulation (i.e., activation, stimulation, increase), or down regulation (i e , inhibition, suppression, reduction, or decrease) of a response, or the two in combination or apart.

As used herein a “modulator” of the level or activity of a DNA repair enzyme or of a gene encoding a DNA repair enzyme includes an activator and/or inhibitor of that gene or polypeptide and is used to refer to compounds that activate or inhibit the level of expression of the gene or polypeptide or an activity of the gene or polypeptide.

As used herein, the term “mutation” means a change in a nucleic acid sequence (in comparison to a wild-type or normal nucleic acid sequence) that alters or eliminates the function of an encoded polypeptide, that alters or eliminates the amount of an encoded polypeptide produced, or that alters or eliminates a regulatory function of the nucleic acid having acquired a mutation. Mutations include, but are not limited to, point mutations, deletions, insertions, inversions, duplications, single-stranded and double-stranded DNA breaks, etc. as known in the art.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, “polypeptide” and “protein” are used interchangeably herein to refer to a polymer of amino acid residues. The terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. Preferred polypeptides are DNA repair enzymes.

As used herein, “subject” or “patient” to be treated for a pathological condition, disorder, or disease by a subject method means either a human or non-human animal in need of treatment for a pathological condition, disorder, or disease.

As used herein, the terms “treat”, “treating”, and “treatment” include: (1) preventing a pathological condition, disorder, or disease, i.e. causing the clinical symptoms of the pathological condition, disorder, or disease not to develop in a subject that may be predisposed to the pathological condition, disorder, or disease but does not yet experience any symptoms of the pathological condition, disorder, or disease; (2) inhibiting the pathological condition, disorder, or disease, i.e. arresting or reducing the development of the pathological condition, disorder, or disease or its clinical symptoms; or (3) relieving the pathological condition, disorder, or disease, i.e. causing regression of the pathological condition, disorder, or disease or its clinical symptoms. These terms encompass also prophylaxis, therapy and cure. Treatment means any manner in which the symptoms of a pathological condition, disorder, or disease are ameliorated or otherwise beneficially altered. Preferably, the subject in need of such treatment is a mammal, more preferable a human.

### II. Compositions and Methods

The small molecule therapeutic agents, referred to herein sometimes as Yel compounds, and their analogs are intended to mitigate, treat, or ameliorate a DNA repair deficiency disorder or a symptom associated with a DNA repair deficiency disorder. The DNA repair deficiency disorders, include, but are not limited to Ataxia Telangiectasia (A-T), Xeroderma Pigmentosum (XP), Fanconi's Anemia (FA), Li Fraumeni syndrome, Nijmegen breakage syndrome (NBS), A-T-like disorder (ATLD), Werner's syndrome, Bloom's syndrome, Rothmund-Thompson syndrome, Cockayne's syndrome (CS), Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, Ataxia with oculomotor apraxia 2, Diamond Blackfan anemia, Rapadilino syndrome, Turcot Syndrome, Seckle Syndrome, Lynch syndrome, NBS-like syndrome, RIDDLE Syndrome, and others.

The agents are a novel approach to ameliorating the symptoms of DNA repair deficiency disorders by upregulating DNA repair mechanisms. Yel002 treatment induces an enrichment in components of ATM Signaling, including components of DNA double-stranded break (DSB) repair by homologous and non-homologous end joining Among the components are ATM, MRE11, NBN, and RAD50. Additionally, Yel002 treatment induces an increase in the abundance of proteins that directly affect DNA damage repair via detection, excision repair and ligation. These proteins include, but not limited to, APEX1 (APEX nuclease 1), DDB1 (Damage-specific DNA binding protein 1), and XRCC4. Yel002 treatment also induces an increase in the components of the structural maintenance of chromosomes (SMC) proteins, which are key components of complexes that have a variety of cellular functions involving chromosome structure and remodeling during the cell cycle and DNA damage repair. These SMC proteins include, but are not limited to, SMC3, a component of Cohesin, and SMC2 and 4, and components of Condensin.

Without functional ATM, Yel002 exerts effects on many DNA repair proteins involved in replication, recombination, or homologous recombination. These include AKT1, BAX, BAG1, ARF1, CDK1/2/4, DAPS, BSG, H-RAS, RAC1, S11, and REL. There is also an upregulation of mTOR signaling components AKT, HRAS, R-RAS, MAPK1, RAC1, and RHO A/C/G/J/T2.

Yel compounds are small biologically active molecules that, among other routes of administration, can either be injected or administered in an oral form. Details are described herein.

Injection and/or oral administration are preferred modes of administration.

Animal testing demonstrated improvement in relevant health indicators (weight increase, lack of associated cancers, increase in lifespan) along with limited toxicity data following regular administration of Yel compounds, in particular, Yel002 (also sometimes referred to herein as “Yel002”).

Of course, variations, changes, modifications and substitution of equivalents in the disclosed embodiments will become apparent to those of ordinary skill in the art upon reading the foregoing description. The inventors expect skilled artisans to employ such variations, changes, modifications and substitution of equivalents as appropriate, and the inventors intend for the invention to be practiced otherwise than specifically described herein. Those of skill in the art will readily recognize a variety of non-critical parameters that could be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

One aspect of the invention provides a compound, which is effective at mitigating, treating, or ameliorating a DNA repair deficiency disorder or a symptom associated with a DNA repair deficiency disorder. The compound can be a synthetic compound or a natural product in a substantially purified form. The compound also includes a pharmaceutically acceptable salt thereof, a prodrug thereof, a hydrate thereof, a solvate thereof, or a polymorphic crystal thereof.

In some embodiments of the compound, the compound has a general structure of

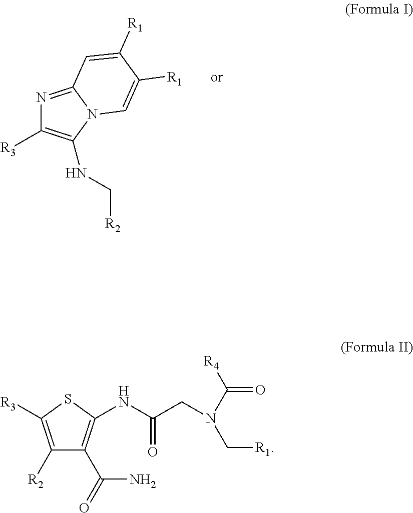

In Formula I, each R₁, R₂ and R₃ are independently hydrogen, straight chain or branched C1-C20 alkyl, alkenyl, or alkynyl, which is substituted or unsubstituted, cyclo alkyl, cyclo alkenyl, heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, alkoxy, or acyl groups along with a list of the following exemplary substitutions:

Each R₁ independently=one or more of the following NH₂, OH, OMe, Me, H, CH₂OH, BH₂, SMe,

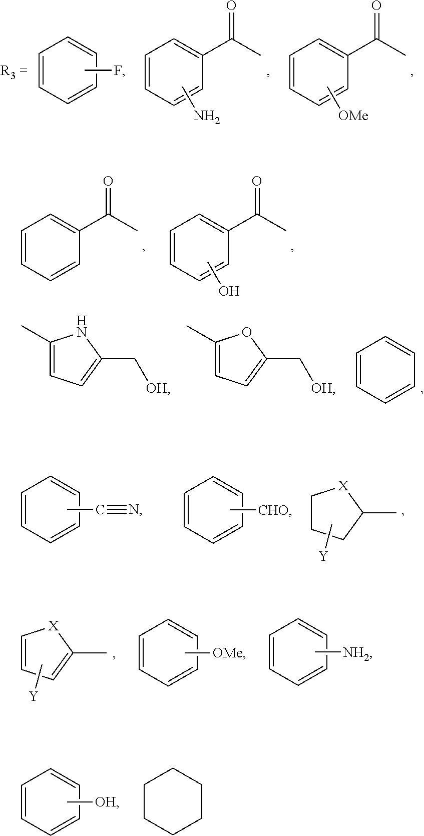

X=S, HN, O, O, BH, CH₂;

Y=NH₂, OH, OMe, Me, H, CH₂OH, BH₂, SeMe, SMe

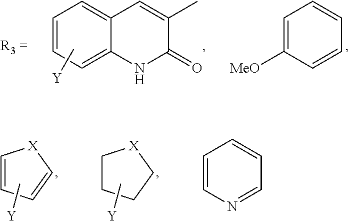

X=S, HN, O, BH, CH₂

Y=NH₂, OH, OMe, Me, H, CH₂OH, BH₂, ScMe, SMe

In Formula II, R₁, R₂, R₃, and R₄ are independently hydrogen, straight chain or branched C1-C20 alkyl, alkenyl, or alkynyl, which is substituted or unsubstituted, cyclo alkyl, cyclo alkenyl, heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, alkoxy, or acyl groups along with a list of the following exemplary substitutions:

### R₁=R₄ and are:

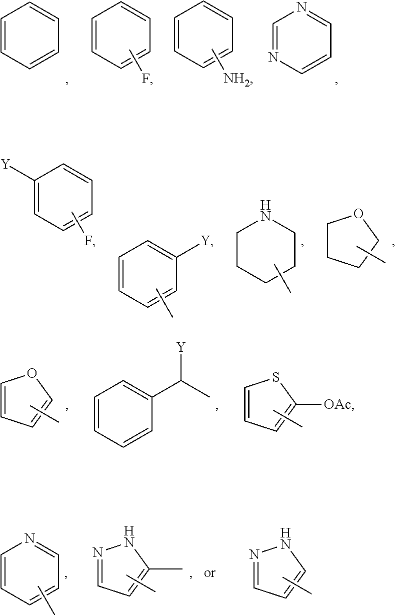

### R₂=R₃=and are CH₃, OH, O, SH, H, NH₂, or

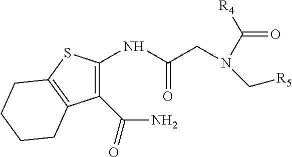

In some embodiments of the compound, the compounds of formula I can have a Tanimoto coefficient 0.7 or above, based on compound of formula IA:

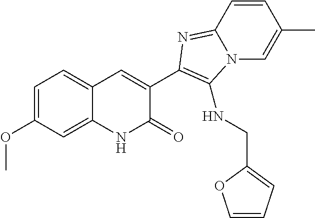 [SMILES: COc1ccc2cc(-c3nc4ccc(C)cn4c3NCc3ccco3)c(=O)[nH]c2c1]

(Formula IA, also described below as Yel002 or Rad2), and compounds of formula II can have a Tanimoto coefficient Tanimoto coefficient 0.7 or above, based on the compound of formula IIA or formula IIB:

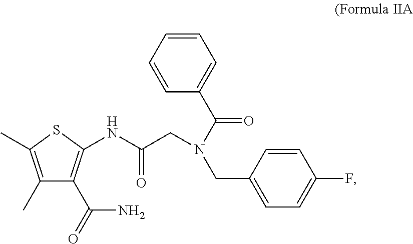 [SMILES: Cc1sc(NC(=O)CN(Cc2ccc(F)cc2)C(=O)c2ccccc2)c(C(N)=O)c1C]

also described below as

Rad I or Yel001)

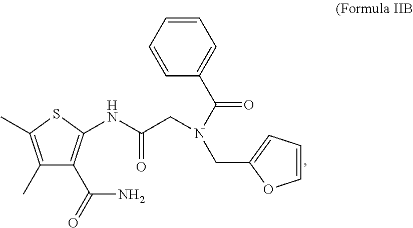 [SMILES: Cc1sc(NC(=O)CN(Cc2ccco2)C(=O)c2ccccc2)c(C(N)=O)c1C]

also described below as CJ010).

In some embodiments, in Formula I:

R₁ is a short chain alkyl, such as methyl, ethyl, an alkenyl, or phenyl;

R₂ is an alkyl, alkenyl, or aryl, such as phenyl, or 2-furanyl; and

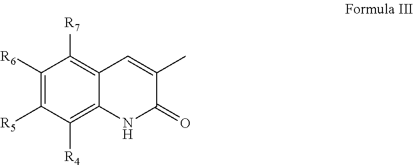

R₃ is a group having formula III, where R₄, R₅, R₆, and R₇ are independently H, short chain alkyl such as methyl, ethyl, propyl, isopropyl, n-butyl, or t-butyl, phenyl, short chain alkoxy, such as methoxy, or ethoxy, phenoxy, halo (F, Cl, Br, or I), or amino groups. In some embodiments, in formula III, R₄, R₆, and R₇ are hydrogen, and R₅ is methoxy.

In some embodiments of the compound, in the compound of Formulae I, R₁, R₂, R₃, R₄, R₅, R₆, and R₇ are selected such that compound of Formula IA is specifically 5 excluded from the definition of the compound of Formula I.

In some embodiments, the compound is an analogue of Formula IA selected from Formulae IB-IH.

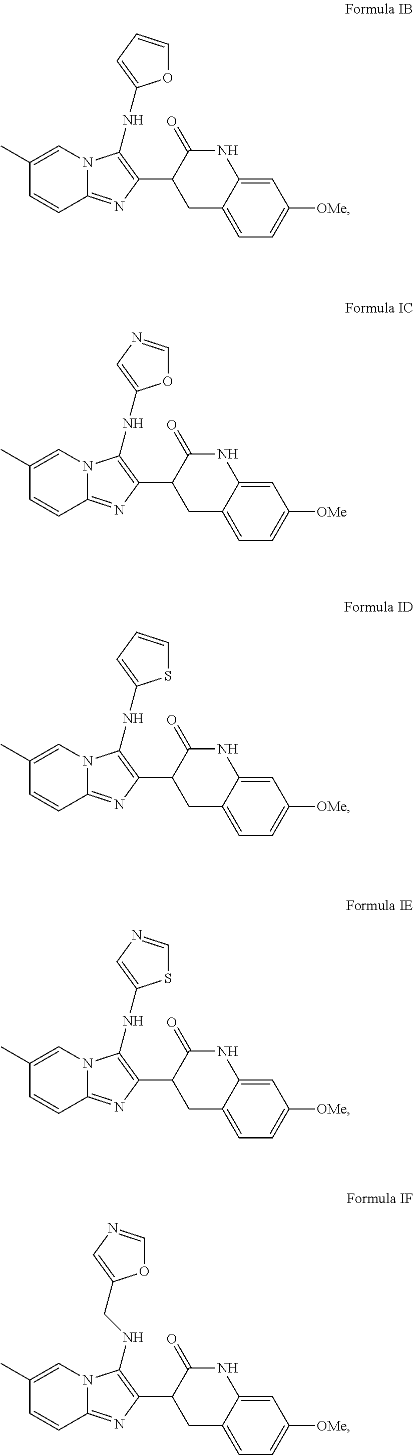 [SMILES: COc1ccc2c(c1)NC(=O)C(c1nc3ccc(C)cn3c1Nc1ccco1)C2 ; COc1ccc2c(c1)NC(=O)C(c1nc3ccc(C)cn3c1Nc1cnco1)C2 ; COc1ccc2c(c1)NC(=O)C(c1nc3ccc(C)cn3c1Nc1cccs1)C2 ; COc1ccc2c(c1)NC(=O)C(c1nc3ccc(C)cn3c1Nc1cncs1)C2 ; COc1ccc2c(c1)NC(=O)C(c1nc3ccc(C)cn3c1NCc1cnco1)C2]

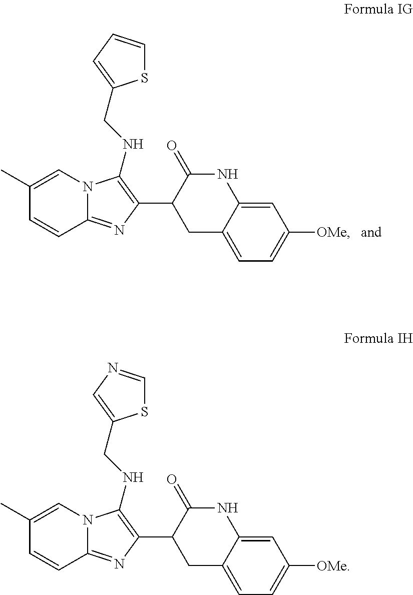 [SMILES: COc1ccc2c(c1)NC(=O)C(c1nc3ccc(C)cn3c1NCc1cccs1)C2 ; COc1ccc2c(c1)NC(=O)C(c1nc3ccc(C)cn3c1NCc1cncs1)C2]

In some embodiments of the compound, in the compound of Formulae II, R₁, R₂, R₃, and are selected such that compound of Formula IIA is specifically excluded from the definition of the compound of Formula II.

In some embodiments of the compound, it is a natural product in a substantially purified form. In some embodiments, the compound has a Formula I or Formula II, both defined above. As used herein, the term “substantially purified” refers to a purity of about

70% or higher, about 80% or higher, about 90% or higher, about 95% or higher, about 99% or higher.

Further, the compounds of the present invention include hydrates thereof, various pharmaceutically acceptable solvates thereof, and polymorphic crystals thereof.

A compound disclosed herein can be isolated from a natural source or prepared according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Exemplary methods of making the compound is provided in the general section of the Examples, described below.

In another aspect of the present invention, it is provided a composition, which composition comprising a compound of the various embodiments disclosed herein. The composition comprises the compound in an amount effective for mitigating tissue damage or lethality induced by an agent. In some embodiments, the composition includes a compound in an effective amount for mitigating, treating, or ameliorating a DNA repair deficiency disorder. In some embodiments of the composition, the composition can further optionally include at least one other therapeutic agent.

In some embodiments of the composition, the composition of various embodiments disclosed herein further comprises an excipient.

In some embodiments of the composition, the composition of various embodiments disclosed herein further comprises a pharmaceutically acceptable carrier.

The composition of various embodiments disclosed herein can be formulated into a formulation for local delivery or systemic delivery. In some embodiments, the composition is formulated into a formulation for oral administration, intravenous injection, injection, topical administration, implant, or pulmonary administration.

In a further aspect of the present invention, it is provided a method of screening for a compound effective as a mitigating agent. The method comprises:

generating a screening system capable of screening a compound against a DNA-repair deficiency disorder; and

subject a compound to the screening, and

identifying a candidate compound if the compound significantly reduces genetic instability, induces DNA repair, restores proliferative regulation in stem cells, restores cell viability in bone marrow, increases the number of red blood cell progenitors, or increases the production of erythrocytes as compared to a control.

In some embodiments of the method, the compound has a structure of Formula I or Formula II.

In a further aspect of the present invention, it is provided a method of preparing a composition. The method comprises providing a compound which is effective for mitigating, treating, or ameliorating a DNA repair deficiency disorder or a symptom associated with a DNA repair deficiency disorder, and forming the composition of the various embodiments disclosed herein. The compound is as in the various embodiments disclosed herein.

In a further aspect of the present invention, it is provided a method of treating, preventing, or ameliorating a condition. The method comprises administering to a subject a compound or a composition according to the various embodiments of disclosed herein.

As used herein, the term “pharmacologically acceptable salt” is not specifically limited as far as it can be used in medicaments. Examples of a salt that the compound of the present invention forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

As used herein, the term “prodrug” shall mean a precursor (forerunner) of a drug. A prodrug must undergo chemical conversion by metabolic processes before becoming an active pharmacological agent.

As used herein, the term “mitigation” or “amelioration” shall mean, for example, a reduction of cell killing, reduction in genetic instability, increase in DNA repair, restoration of proliferative regulation in stem cells, restoration of cell viability in bone marrow, increase in the number of red blood cell progenitors, and increase erythrocyte production by a percentage of 5% or higher, 10% or higher, 25% or higher, 50% or higher, 75% or higher, 100% or higher, 200% or higher, 500% or higher, or 1000% or higher as compared to a control.

As used herein, the terms “Yell”, “Yel001”, and “Radl” are used interchangeably.

As used herein, the terms “Yel002”, “Yel002”, and “Rad2” are used interchangeably.

Tanimoto coefficient has been widely used in the art of design and preparation of compounds with similar physical, chemical, and pharmacological properties (see, Ajay Kumar, et al., Computational Approach to Investigate Similarity in Natural Products Using Tanimoto Coefficient and Euclidean Distance, The MP Journal of Information Technology, Vol. 6, No. 1, pp. 16-23, March 2010; Gen Kawamura, Shigeto Seno, Yoichi Takenaka and Hideo Matsuda: “A Combination Method of the Tanimoto Coefficient and Proximity Measure of Random Forest for Compound Activity Prediction”, IPSJ Digital Courier, Vol. 4, pp.238-249. (2008)).

### Formulations

The composition disclosed herein can be formulated into various formulations. The composition can be formulated for systemic or local delivery of the radiation protective compound. For example, such formulations include, e.g., liquid, solid, or semi-solid formulations for various mode of administration, e.g., oral administration, subcutaneous injection, intravenous injection, topical administration, or implant.

The compositions can be formed into a formulation suitable for a desired mode administration. In some embodiments, the composition can include a pharmaceutically acceptable carrier. The content of the compound disclosed herein in the composition according to the present invention may range, but is not limited to, preferably from 0.001 to 20 wt. %, more preferably from 0.01 to 15 wt. %, most preferably from 0.05 to 10 wt. %.

Formulations can be made suitable for different routes of administration, for example, liquids for intravenous administration, topical administration via application to the surface of the diseased site, or mucosal application to cavities of the nose, mouth, eye, rectum, vagina or bronchopulmonary; solid dosage forms that may dissolve in the mouth or be inhaled through the bronchopulmonary; and semisolids that may be applied to cavity surfaces of the nose, mouth, eye, rectum, or vagina.

Examples of the carrier employed in the composition disclosed herein can include any desired carriers generally contained in drugs, fibers, polymeric materials and the like. Concerning pharmaceutical compositions, illustrative of such desired carriers are excipients, coloring matters, taste or smell corrigents, binders, disintegrators, coating materials, stabilizers, pH regulators, sugar-coating materials, emulsifiers, dispersants, and solubilizers. Especially for external dermal preparations, illustrative examples can include hydrocarbons such as liquid paraffin and vaseline, esters such as spermaceti and bees wax, triglycerides such as olive oil and beef tallow, higher alcohols such as cetanol and oleyl alcohol, fatty acids such as stearic acid and oleic acid, polyhydric alcohols such as propylene glycol and glycerin, nonionic surfactants, anionic surfactants, cationic surfactants, and thickeners. For clothing and plastics, illustrative examples can include plasticizers, crosslinking agents, coloring matters, antioxidants, and ultraviolet absorbers.

In some embodiments, an aqueous preparation or formulation of the composition disclosed herein may contain buffers, surfactants, humectants, preservatives, flavorings, stabilizers (including antioxidants) colorants, and other additives used in preparations administered into the oral cavity.

In some embodiments, liquid compositions preferably should have a pH value ranging from 2 to 10, preferably 3.5 to 9, most preferably 4 to 8. A preparation having a pH of less than 4 would be likely to cause a stinging sensation. Furthermore, the preparations having a higher pH are often unpleasant to use. The active agents need not be in solution to be effective. The active agents may be present wholly or in part as suspensions in aqueous solutions used as carriers to provide liquid compositions. The preparations are buffered as necessary to provide the appropriate pH.

Appropriate buffer systems include citric acid-citrate salts, acetic acid-acetate salts, and benzoic acid-benzoic salt systems. However, any buffer system commonly used for preparing medicinal compositions would be appropriate. While the vehicle used generally is primarily water, other vehicles may be present such as alcohols, glycols (polyethylene glycol or polypropylene glycol are examples), glycerin, and the like may be used to solubilize the active agents. Surfactants may include anionic, nonionic, amphoteric and cationic surfactants which are known in the art as appropriate ingredients for mouthwashes.

Liquid formulations may contain additional components to improve the effectiveness of the product. Fox example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, and gellan gums. Gellan gums are preferred since aqueous solutions containing certain gellan gums may be prepared so that they will experience an increase in viscosity upon contact with electrolytes.

Some examples of the formulations of the composition disclosed herein include, for example, solid formulations such as tablets, capsules, granules, pills, troches, powders or suppositories, or liquid formulations such as syrups, elixirs, suspensions or injections, as well as aerosols, eye drops, ointments, ophthalmic ointments, emulsions, creams, liniments or lotions. These formulations may be prepared in accordance with conventional methods commonly used in the field of drug formulations.

In some embodiments, various additives which are commonly used in the drug formulation field, can be used Such additives include, for example, saccharides such as lactose or glucose, a starch such as corn, wheat or rice, a vegetable oil such as soybean oil, peanuts oil or sesame oil, a fatty acid such as stearic acid, an inorganic salt such as magnesium metasilicate aluminate or anhydrous calcium phosphate, a synthetic polymer such as polyvinylpyrrolidone or polyalkylene glycol, a fatty acid salt such as calcium stearate or magnesium stearate, an alcohol such as stearyl alcohol or benzyl alcohol, a synthetic cellulose derivative such as methyl cellulose, carboxymethyl cellulose, ethyl cellulose or hydroxy-propylmethyl cellulose, or others such as water, gelatin, talc and gum arabic.

Further, in the case of a liquid formulation, it may be in such a form that at the time of use, it is dissolved or suspended in water or in other suitable medium. Especially when administration is carried out by, e.g., intramuscular injection, intravenous injection or subcutaneous injection, a suitable medium for such an injection may, for example, be distilled water for injection, a hydrochloric acid lidocaine aqueous solution (for intramuscular injection), physiological saline, an aqueous glucose solution, ethanol, liquid for intravenous injection (such as an aqueous solution of citric acid and sodium citrate) or an electrolyte solution (for intravenous drip and intravenous injection), or a mixed solution thereof. Further, a buffer or a preservative may be added.

In some embodiments, for delivery into a cell, the composition disclosed herein can be formulated into liposomal preparations (e.g., liposomal suspensions or particles). Liposomal suspensions (including liposomes targeted to infected cells with monoclonal antibodies to viral antigens) are also preferred as pharmaceutically acceptable carriers. Methods for encapsulation or incorporation of compounds into liposomes are described by Cozzani, I.; Joni, G.; Bertoloni, G.; Milanesi, C.; Sicuro, T. Chem. Biol. Interact. 53, 131-143 (1985) and by Joni, G.; Tornio, L.; Reddi, E.; Rossi, E. Br. S. Cancer 48, 307-309 (1983). These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Joni, G.; Reddi, E.; Cozzani, Tornio, L. Br. J. Cancer, 53(5), 615-21 (1986).

These formulations may contain usually from 0.001 to 100 wt %, preferably from 0.005 to 100 wt %, of the active ingredient in the case of the above-mentioned solid formulations, and may contain from 0.05 to 10 wt %, preferably from 1 to 5 wt %, in the case of other formulations.

A practically preferred dose of the compositions disclosed herein varies depending upon the type of the compound used, the type of the composition blended, the sex, age, weight, diseased degree and the particular section to be treated of the patient, but it is usually from 0.1 to 150 mg/kg in the case of oral administration and from 0.01 to 150 mg/kg in the case of parenteral administration, per adult per day. The number of times of administration varies depending upon the administration method and the symptom, but it is preferred to carry out the administration from one to five times per day.

As used herein, the terms “formulation” and “preparation” are used interchangeably.

### Other Therapeutic Agents

In some embodiments, the compound disclosed herein can combine with one or more other therapeutic agents so as to provide combinatorial treatment. Such other therapeutic agents that can be combined with the compound disclosed herein include, but are not limited to, Amifostine, free radical scavengers, growth factors, immune modulators, anti-apoptotic agents, capture agents et cetera such as Tempol, CBL502, tetracyclin and analogs.

In some embodiments, the compound disclosed herein can be used with NAC (N-acetylcysteine) or a mixture of vitamins C and E, a-lipoic acid, coenzyme Q10, N-acetylcysteine (NAC) and 1-selenomethionine (SEM).

### Method of Use

The compound or composition disclosed herein can be used for treating, mitigating, or ameliorating various conditions pertaining to DNA repair deficiency disorder. For example, the compound or composition disclosed herein can be used for mitigation, treatment, or amelioration of a DNA repair deficiency disorder.

The invention method of use generally comprises administering to a subject (e.g., a human being) a compound or a composition disclosed herein. Such administering can be local administration or systemic administration, which administering can be achieved by, for example, oral administration, subcutaneous injection, intravenous injection, topical administration, or implant.

Examples of conditions related to DNA repair deficiency disorders include, but are not limited to, Ataxia Telangiectasia (A-T), Xeroderma Pigmentosum (XP), Fanconi's Anemia (FA), Li Fraumeni syndrome, Nijmegen breakage syndrome (NBS), A-T-like disorder (ATLD), Werner's syndrome, Bloom's syndrome, Rothmund-Thompson syndrome, Cockayne's syndrome (CS), Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, Ataxia with oculomotor apraxia 2, Diamond-Blackfan anemia, Rapadilino syndrome, Turcot Syndrome, Seckle Syndrome, Lynch syndrome, NBS-like syndrome, and RIDDLE Syndrome and others like those.

As can be appreciated from the disclosure above, the present invention has a wide variety of applications. The invention is further illustrated by the following examples, which are only illustrative and are not intended to limit the definition and scope of the invention in any way.

### EXAMPLES

The embodiments of the present invention are illustrated by the following set forth examples. All parameters and data shall not be construed to limit the scope of the embodiments of the invention.

### Example 1

### Ataxia Telangiectasia Mouse Models Demonstrate the Benefits of Yel002 Administration as a Lone-Term Treatment

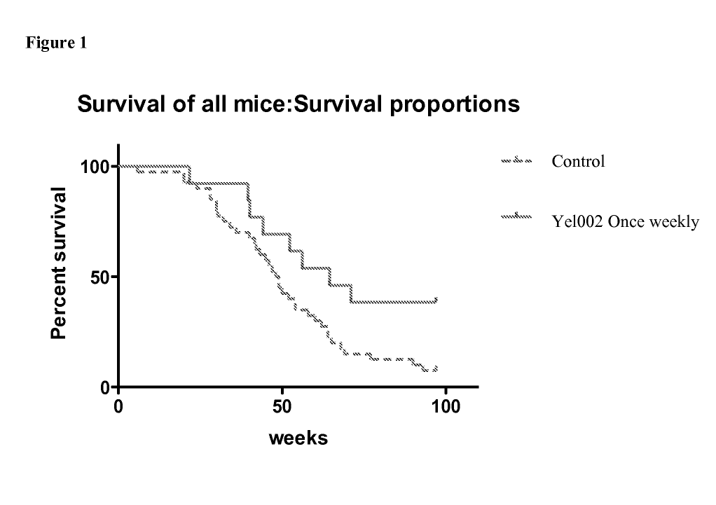

13 mice defective in ATM protein production were injected once weekly with Yel002. Yel002 (75mg/kg in saline carrier) was administered subcutaneously once weekly beginning as soon as mice were identified as homozygous defective by genotyping (at ˜1 month of age.). The ongoing study showed a significant (p<0.05) reduction in death rate when compared to lifespan data of untreated−/− mice of the same line kept in the same facility (FIG. 1). The difference in life expectancy is 16 weeks which translates into 12 years for a human patient which would be a tremendous relieve of people with AT.

### Example 2

### Mechanism of Action Studies on Normal and ATM-Deficient Cells Reveal Upregulation of DNA-Repair Machinery Following Yel002 Administration

### High-Through Proteomics Reveals MOA

Flasks of ATM-deficient LCLs and Normal LCLs were incubated with PBS or Yel002 for two hours. Specified flasks were exposed to 5 Gy y- irradiation, and then cells from all experimental flasks were harvested after 24 hours. Cells were lysed and proteins extracted, solubilized, and digested by eFASP with 0.2% DCA. The resulting peptides from eFASP were separated based on isoelectric point and hydrophilicity using ERLIC on a polyWAX LP column. Fractions were pooled based on the 215 nm UV-Vis absorbance trace, yielding 25 to 30 fractions. Each fraction was injected onto a reverse phase 219 column and separated by UPLC on a nanoACQUITY. Eluting peptides were analyzed by LC-MSE on a Synapt HDMS. Raw data files produced for all fractions of a sample were merged and searched using PLGS. DECO was utilized to correct peptide ion volume data for any peptide found in multiple ERLIC fractions.

For comparative analysis, each processed data set was compared in a head-to-head fashion using Expression Analysis for normalization, ratio determination, and statistical analysis of expression differences. All ratios for N LCLs or AT LCLs were determined with protein abundance for the IR or the YEL002 condition as the numerator, and the protein abundance for the PBS control as the denominator. The threshold for protein upregulation was a natural log ratio greater than 0.45, with a p-value of upregulation greater than 0.95 (See, Table 1).

TABLE 1
Summary of differentially regulated proteins in each condition,
for each cell line.

| | |
|---|---|
| N24 | AT24 |

| | | YEL002/ | | YEL002/ |
|---|---|---|---|---|
| | IR/PBS | PBS | IR/PBS | PBS |
| Upregulated | 238 | 244 | 143 | 307 |
| Unique | 418 | 341 | 473 | 470 |

Expression analysis enabled the head-to-head comparisons between treated and control in each cell line. The threshold for protein upregulation was a natural log ratio greater than 0.45, with a p-value of upregulation greater than 0.95. Unique identifications must have a PLGS score greater than 100.

Proteins flagged as unique in a condition must have been identified with a PLGS score over 100. In most cases, unique protein identifications had PLGS score well over 1,000. For comparisons between each condition in the N24 LCLs and the AT24 LCLs, the ratios were determined with the protein abundance from the AT24 LCL as the denominator (See Table 2).

TABLE 2
Summary of differentially regulated proteins in
each condition, between the cell lines.

| | N24/AT24 | | |
|---|---|---|---|
| | PBS | IR | YEL002 |
| Upregulated | 307 | 198 | 275 |
| Downregulated | 397 | 170 | 228 |
| Unique N24 | 399 | 474 | 378 |
| Unique AT24 | 336 | 508 | 665 |

Expression analysis enabled the head-to-head comparison of protein abundance between cell lines, for each condition. The threshold for protein upregulation was a natural log ratio greater than 0.45, with a p-value of upregulation greater than 0.95. The threshold for protein upregulation was a natural log ratio less than -0.45, with a p-value of downregulation less than 0.05. Proteins unique to either cell line must have a PLGS score greater than 100.

The threshold for upregulation was the same, and the threshold for downregulation was a natural log ratio less than -0.45, with a p-value of downregulation less than 0.05. Ingenuity pathway analysis of the differentially expressed proteins from the head-to-head comparisons allows for the determination of wide proteomic effects of IR and YEL002, and their dependence on ATM. Additionally, we can compare the cell lines without experimental conditions, to explore the proteomic consequences of ATM-deficiency.

After YEL002 treatment there is enrichment in components of ATM Signaling (p-value=5.75E-05), including components of DSB repair by homologous recombination (p-value=7.08E-04) and non-homologous recombination (p-value=4.47E-05). Among the most important are ATM (N24YEL002, PLGS score 604), MREl l (In ratio N24+PBS/N24+YEL002=0.42, p-value=1.00), NBN (N24YEL002, PLGS score 678), and RAD50 (N24YEL002, PLGS score 713). In the variety of experiments Applicants have performed, these components are rarely identified due to low copy number, especially ATM. Additionally, YEL002 treatment induces an increase in the abundance of proteins that directly affect DNA damage repair via detection, excision repair and ligation. These proteins include APEX1, DDB1, and XRCC4.

Structural maintenance of chromosomes (SMC) proteins are key components of complexes that have a variety of cellular functions involving chromosome structure and remodelling during the cell cycle and DNA damage repair 45. SMC3, a component of Cohesin, and SMC2 and 4, components of Condensin, are upregulated following YEL002 treatment.

Without functional ATM, Yel002 exerts effects on many DNA repair proteins involved in replication, recombination, or homologous recombination. These include AKT1, BAX, BAG1, ARF1, CDK1/2/4, DAPS, BSG, H-RAS, RAC1, S11, and REL. There is also an upregulation of mTOR signaling components AKT, HRAS, R-RAS, MAPK1, RAC1, RHO A/C/G/J/T2.

All publications, including but not limited to patents and patent applications, cited in this specification, to the extent that they provide exemplary procedural or other details supplementary to those set forth herein, are specifically incorporated herein by reference as if each individual publication were specifically and individually indicated to be incorporated by reference herein as though fully set forth.

### Equivalents

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A method for mitigating, treating or ameliorating in a patient a DNA repair deficiency disorder or a symptom associated with a DNA repair deficiency disorder, the method comprising the step of:
(a) administering to a subject in need of mitigating, treating or ameliorating a DNA repair deficiency disorder or a symptom associated with a DNA repair deficiency disorder a pharmaceutically effective amount of a composition comprising:
(i) a compound having Formula 1; or
(ii) a compound having Formula 2; or
(iii) a pharmaceutically acceptable salt or prodrug of (i) and/or (ii).

2. The method of claim 1, wherein the DNA repair deficiency disorder is selected from Ataxia Telanglectasia (A-T), Xeroderma Pigmentosaum (XP), Fanconi's Anemia (FA), Li Fraumeni syndrome, Nijmegen breakage syndrome (NBS), A-T-like disorder (ATLD), Werner's syndrome, Bloom's syndrome, Rothmund-Thompson syndrome, Cockayne's syndrome (CS), Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, or Ataxia with oculomotor apraxia 2, Diamond-Blackfan anemia, Rapadilino syndrome, Turcot Syndrome, Seckle Syndrome, Lynch syndrome, NBS-like syndrome, or RIDDLE Syndrome.

3. A method for modulating a level or activity of a DNA repair enzyme or a level or activity of gene encoding a DNA repair enzyme, the method comprising the step of:
(a) administering to a subject in need of modulating level or activity of a DNA repair enzyme or a level or activity of gene encoding a DNA repair enzyme a pharmaceutically effective amount of a composition comprising:
(i) a compound having Formula 1; or
(ii) a compound having Formula 2; or
(iii) single stereoisomer, mixtures of stereoisomers, pharmaceutically acceptable salts or prodrugs of (i) and/or (ii).

4. The method of claim 3, wherein the DNA repair enzyme is selected from ATM, MRE11, NBN, RAD50. APEX1 (APEX nuclease 1), DDB1 (Damage-specific DNA binding protein 1), XRCC4, SMC3, SMC2, SMC4, or condensin.

5. The method of claim 1, wherein the compound is Yel002.

6. The method of claim 1, wherein the compound comprises the structure of Formula I or Formula II: 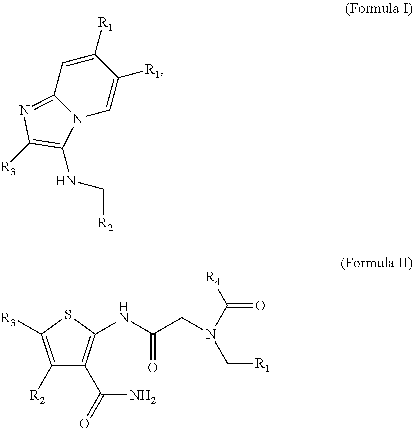
wherein:
in Formula I, each R₁, R₂ and R₃ are independently hydrogen, straight chain or branched C1-C20 alkyl, alkenyl, or alkenyl, which is substituted or unsubstituted, cyclo alkyl, cyclo alkenyl, heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, alkoxy, or acyl groups; and
in Formula II, R₁, R₂, R₃, and R₄ are independently hydrogen, straight chain or branched C1-C20 alkyl, alkenyl, or alkenyl, which is substituted or unsubstituted, cyclo alkyl, cyclo alkenyl. heterocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfonyl, —SO₄H, alkoxy, or acyl groups
or a pharmaceutically acceptable salt, a hydrate, a solvate, a polymorphic crystal or a prodrug thereof.

7. The method of claim 6, wherein in Formula I, each R₁, independently is one or more of the following NH₂, OH, OMe, Me, H, CH₂OH, BH₂, SMe; 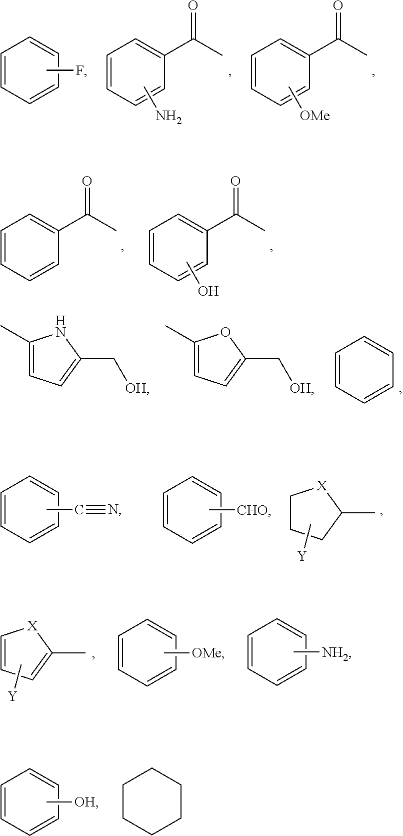
X=S, HN, 0, BH, CH₂;
Y=NH₂, OH, OMe, Me, H, CH₂OH, BH₂, SeMe, SMe; 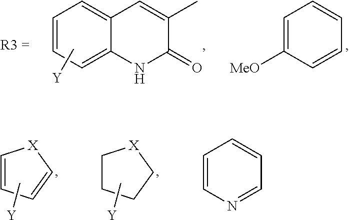
X=S, HN, 0, BH, CH₂,
Y=NH₂, OH, OMe, Me, H, CH₂OH, BH₂, SeMe, SMe;
and in Formula II,
R₁ and R₄ are: 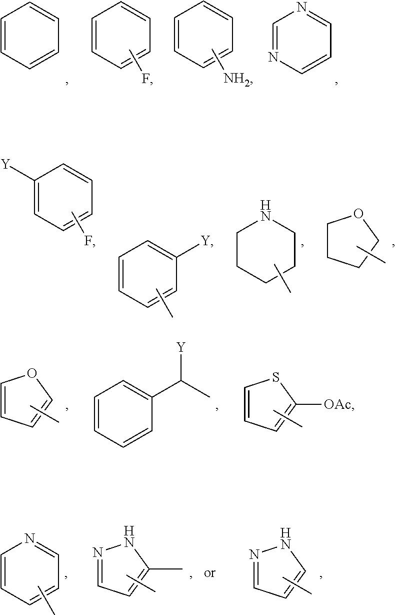
Y=CH₃, OH
and
R₂=R₃=and are CH₃, OH, O, SH, H, NH₂, or

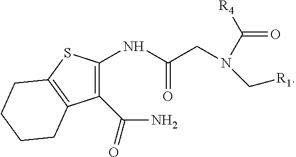

8. The method of claim 1, having a structure of compound having a Tanimoto coefficient at least 0.7 or higher based on a compound of Formula IA, Formula IIA or Formula IIB:

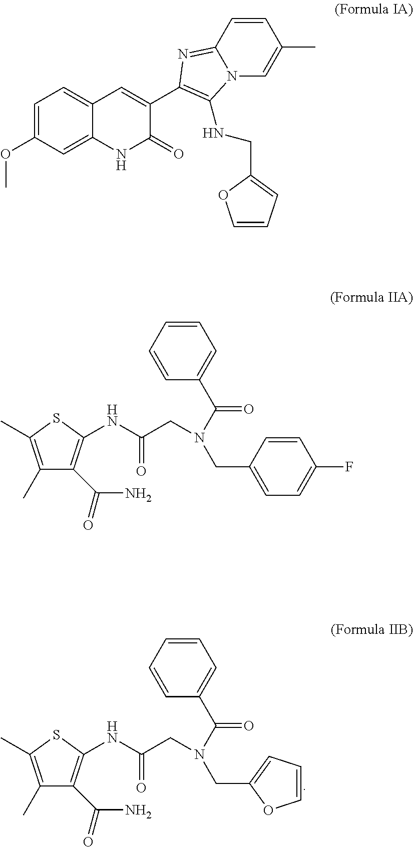

9. The method of claim 1, wherein the compound is selected from: 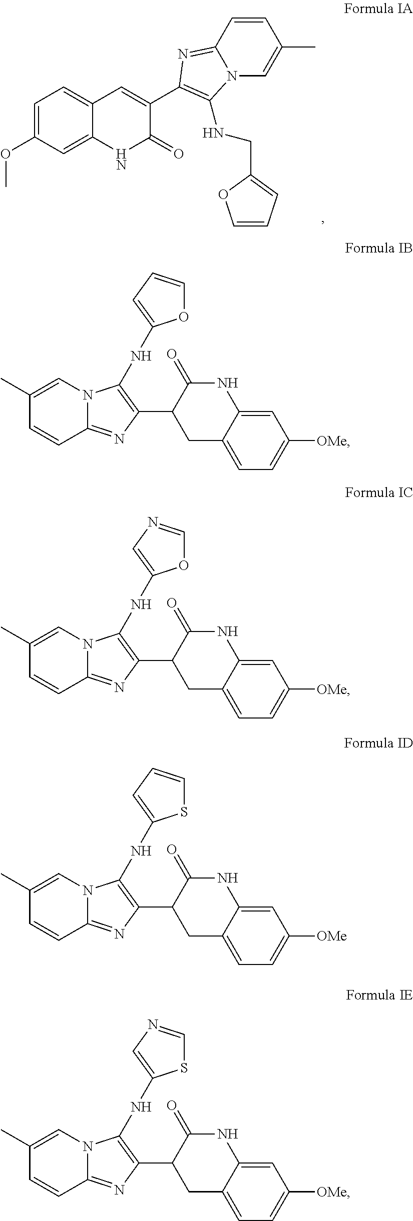 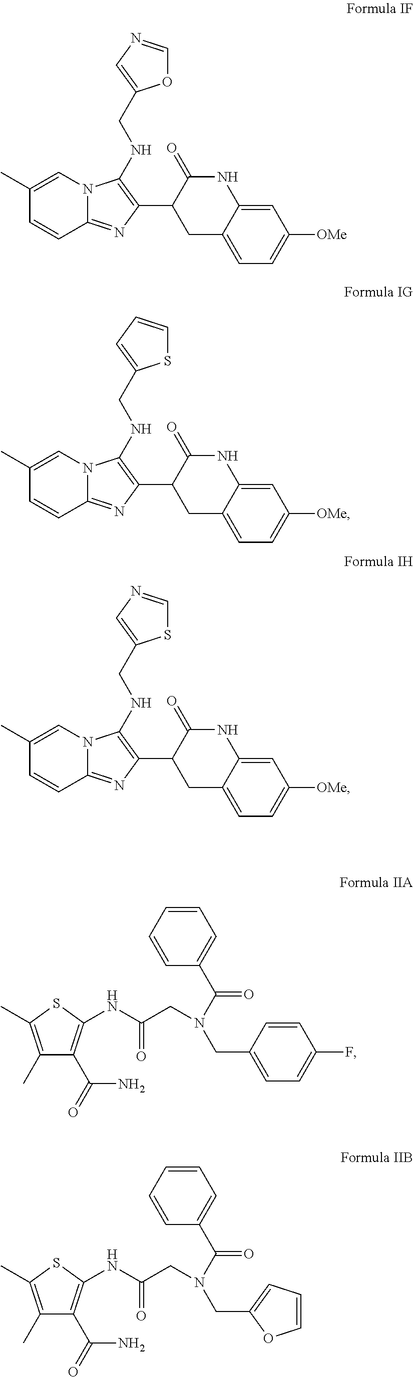 [SMILES: COc1ccc2c(c1)NC(=O)C(c1nc3ccc(C)cn3c1NCc1cnco1)C2 ; COc1ccc2c(c1)NC(=O)C(c1nc3ccc(C)cn3c1NCc1cccs1)C2 ; COc1ccc2c(c1)NC(=O)C(c1nc3ccc(C)cn3c1NCc1cncs1)C2 ; Cc1sc(NC(=O)CN(Cc2ccc(F)cc2)C(=O)c2ccccc2)c(C(N)=O)c1C ; Cc1sc(NC(=O)CN(Cc2ccco2)C(=O)c2ccccc2)c(C(N)=O)c1C]
or combinations thereof.

10. A method of screening for a compound effective as a mitigating agent, the method comprises:
generating a screening system capable of screening a compound against a DNA repair deficiency disorder;
subjecting a compound to the screening, and
identifying a candidate compound if the compound significantly reduces genetic instability, induces DNA repair, restores proliferative regulation in stem cells, restores cell viability in bone marrow, increases the number of red blood cell progenitors, or increases the production of erythrocytes as compared to a control.
